(19) United States
(12) Patent Application Publication
(10) Pub. No.: US 2018/0008837 A1
(43) Pub. Date: Jan. 11, 2018

(54) LIGHT THERAPY DELIVERY DEVICES AND METHODS FOR THE FEMALE GENITALIA AND AREAS PROXIMAL THERETO

(71) Applicant: Jack K. Zhang, West Palm Beach, FL (US)

(72) Inventor: Jack K. Zhang, West Palm Beach, FL (US)

(21) Appl. No.: 15/645,467

(22) Filed: Jul. 10, 2017

Related U.S. Application Data
(60) Provisional application No. 62/493,613, filed on Jul. 11, 2016.

(51) Int. Cl. A61N 5/06; A61N 5/067

(52) U.S. Cl. CPC A61N 5/0603; A61N 2005/067; A61N 2005/0663; A61N 2005/0611; A61N 2005/0651; A61N 2005/063; A61N 2005/0659

(57) ABSTRACT

The present invention relates to light therapy devices and related methods to treat the female genitalia and areas proximal thereto, including the vaginal cavity and vulvar regions in cisgender females or transgender females who have undergone sex reassignment, in particular for post-surgical indications, vaginal rejuvenation, or other medical conditions suitable for treatment with light therapy. The devices and methods are configured to provide a therapeutic amount of low level light therapy to a patient in need of treatment thereof.

### CROSS REFERENCE TO RELATED APPLICATIONS

The application claims priority to U.S. Provisional Patent Application No. 62/493,613, filed Jul. 11, 2016. The disclosure of this application is incorporated herein its entirety by this reference.

### FIELD OF THE INVENTION

The present invention relates to light therapy devices and related methods to treat the female genitalia and areas proximal thereto, including the vaginal cavity and vulvar regions in cisgender females or transgender females who have undergone sex reassignment, in particular for post-surgical indications, vaginal rejuvenation, or other medical conditions suitable for treatment with light therapy. The devices and methods are configured to provide a therapeutic amount of low level light therapy to a patient in need of treatment thereof.

### BACKGROUND OF THE INVENTION

Low level light therapy (“LLLT”) relates, in some aspects, to the exposing of cells or tissue to low levels of red and near infrared (“NIR”) light. When the applied light is laser sourced, LLLT is also known as “low level laser” or “cold laser” therapy, as the power densities used are lower than those needed to produce heating of tissue. In this regard, light application is at relatively low energy densities, typically below about 500 mW, as compared to other forms of laser therapy that are used for ablation, cutting, and thermally coagulating tissue. Other types of LLLT can also apply wavelengths in the blue or ultraviolet regions, especially for treatment of conditions that occur at the skin surface, such as psoriasis or infection. While it was originally believed that effective LLLT required the use of coherent laser light, more recently, light emitting diodes (“LEDs”) have been introduced as an alternative light source. Today, the term “low level light therapy” refers to light generated from both laser and LED sources.

Whether the light source is laser or LED, at the most basic level, LLLT is believed to affect a biological change in living tissue by inducing a photochemical reaction in the cell, a process referred to medically as “photobiomodulation” or “PBM.” From clinical observation, it appears that LLLT can hold a wide range of effects at the molecular, cellular, and tissue levels. In addition, the specific modes of action can vary among different applications. As reported in “Effects of Low-Power Light Therapy on Wound Healing: Laser LED, An Bras. Dermatol. 2014; 89(4): 616-23, the disclosure of which is incorporated in its entirety by this reference, the biological effects of LLLT appear to be largely dependent on the treatment parameters of applied light wavelength(s) and dosage(s). While wavelengths of light and dosage parameters specifically defined for an experimental condition have been elucidated by way of many studies described in this referenced review article, the authors indicate that what constitutes a therapeutic dose across medical indications, patients, and the like remains largely unresolved.

Although LLTT has been recognized to be efficacious to treat a wide variety of indications, LLLT nonetheless remains underutilized as a therapy for several reasons. First, since the underlying biochemical mechanisms are poorly understood today, many use cases remain anecdotal and reported results are largely empirical in nature. Accordingly, for applications where clinical efficacy must be demonstrated, LLLT will generally not be prescribed by a medical healthcare provider. Next, for LLLT to be properly applied to a patient in need of treatment, several variables must be selected and repeatedly monitored throughout the course of the treatment. These parameters include wavelength, energy density, power density, pulse structure, and timing of the applied light, as well as the patient medical indication and any associated patient-specific parameters. Each of these features can contribute to the effectiveness of an applied LLLT treatment. A less than optimal choice of parameters can result in reduced effectiveness of the treatment, or even adverse therapeutic outcomes. The multiplicity of treatment variables that can affect LLLT treatment effectiveness likely contributes to the dearth of unambiguous and repeatable results that allow a LLLT to be prescribed regularly in clinical settings. Indeed, many of the published results on LLLT actually demonstrate negative clinical efficacy, possibly because of an inappropriate choice of light source and dosage for the patients being treated.

There is also evidence to suggest that the effectiveness of the treatment varies greatly in relation to both the energy and power density used: there appears to be upper and lower thresholds for both parameters within which LLLT is effective. Outside of these thresholds, the light is either too weak to have any effect, or is so strong that its harmful effects outweigh its benefits. For example, it has been reported that too low of a dose does not promote biological effects, and doses that are too high result in the inhibition of cellular functions. LLLT can therefore be characterized by a biphasic dose response that indicates a range within which beneficial effects can be generated with light application. Notwithstanding this knowledge, to date, there has been no meaningful resolution of dosage parameters for specific medical indications.

Delivery systems associated with LLLT have not lent themselves to well-defined dose application of LLLT to a patient in need of treatment. Lasers have typically been difficult to implement outside of the clinical setting due to need to generate and reliably deliver an effective dose of laser light to a patient. Moreover, the periodic and longitudinal nature of LLLT treatments has made it generally infeasible to treat people cost effectively. Until recently, lasers and the associated componentry have been expensive and have required mains power to generate suitable energy to power the componentry. In addition, most LLLT treatment modalities would require people to travel regularly to a medical provider's office or a hospital and stay for and return to enough times to affect a full course of LLLT treatment that would result in the intended therapeutic outcome.

Male to female sex reassignment is a highly invasive treatment that requires substantially total reconstruction of a patient's genital areas. Healing time is generally many weeks, and both internal and external granulated tissue is a common side effect, with bleeding of such tissue often occurring up to 6 months after surgery. Granulated tissue is a signal of sub-adequate healing and treatment is indicated. Episiotomies can also generate granulated and bleeding tissue for weeks after childbirth, thus requiring treatment to ensure that scar tissue does not form in and around the perineum. Hemorrhoids can comprise a chronic condition, especially in women with compromised genital areas. Similarly, bladder weakness is a common condition, especially in women who have given birth. Yet further, and as disclosed in US Patent Application No. 20090319008, the disclosure of which is incorporated herein in its entirety by this reference, collagen and elastin included in the sub-mucosal layer covering body cavities contribute to the elasticity and tone of the vaginal cavity. Their breakdown and disruption through, for example, the aging process, birth, the existence of a pathological condition (e.g. inflammation or any other disease or disorder) etc. may result in loss of tightness and flexibility of the wall of the respective body cavity. Application of tissue stimulation technique to the vaginal cavity is emerging as an aesthetic treatment to address such lack of elasticity.

In regards to each of these female genitalia-related indications, there remains need for improvements in providing enhanced healing to the internal and external genital areas of female patients in need of treatment. The present invention provides these and other benefits.

### SUMMARY OF THE INVENTION

In broad constructs, the present invention pertains to light therapy devices and related methods to treat the female genitalia and areas proximal thereto, including the vaginal cavity and vulvar regions in cisgender females or transgender females who have undergone sex reassignment, in particular for post-surgical indications, vaginal rejuvenation, or other medical conditions suitable for treatment with light therapy. The devices and methods are configured to provide a therapeutic amount of low level light therapy to a patient in need of treatment thereof.

In some aspects, the invention can comprise a therapeutic device that is a structure fabricated from an optically clear material suitably configured to be engagable with at least one light guide, wherein the at least one light guide is operationally engaged with a light source configured to dispense one or more doses of low level light therapy (LLLT) to the vaginal cavity of a patient when a substantial portion of the structure is inserted in at least part of the vaginal cavity of the patient. The light source can comprise one or more lasers and/or one or more LEDs. In some aspects, the LLLT dosage applied to the patient is generated by a controller in operational engagement with the light source, wherein the controller is configured to dispense LLLT according to dosage instructions generated by a medical provider.

Yet further, the invention can comprise a flexible structure having an optically clear first side for use proximal to the genitalia of a female patient in need of LLLT, wherein the structure is configured to fit proximal to the external genitalia and to dispense a therapeutic amount of light thereto. The structure can be engageable with at least one light guide, wherein the at least one light guide is operationally engaged with a light source configured to dispense one or more doses of LLLT to the patient. A controller in is operational engagement with the light source, and wherein the controller is configured to dispense LLLT from the flexible structure according to dosage instructions generated by a medical provider.

The insertable structure and the flexible structure can be used together, or each can be used independently. Such usage configurations will be a prescribed, monitored and managed by a medical provider.

Methods of using the each of the vaginal insertable and flexible structures are also provided herein.

Additional advantages of the invention will be set forth in part in the description that follows, and in part will be apparent from the description, or may be learned by practice of the invention. The advantages of the invention will be realized and attained by means of the elements and combination particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 illustrates an implementation of the invention as utilized in a patient in need of treatment whereby an insertable vaginal light therapy device is in use with a light therapy delivery liner, and each is in operational and optical communication with a dosing light source.

FIGS. 2a, 2b, and 2c illustrate different views and componentry associated with an insertable vaginal light therapy device.

FIG. 3 illustrates an implementation of light delivery in and through an implementation of an insertable vaginal light delivery device.

FIG. 4 illustrates an implementation of a light therapy delivery liner in a patient in need of treatment.

FIGS. 5a, 5b and 5c illustrate different views and configurations of a light therapy delivery liner.

FIGS. 6a and 6b illustrate alternative implementations of insertable vaginal light delivery devices.

FIG. 7 illustrates a further implementation of an insertable vaginal light delivery device.

### DETAILED DESCRIPTION OF THE INVENTION

In the following detailed description, reference is made to the accompanying drawings, which form a part hereof, and within which are shown by way of illustration certain embodiments by which the subject matter of this disclosure may be practiced. It is to be understood that other embodiments may be utilized and structural changes may be made without departing from the scope of the disclosure. In other words, illustrative embodiments and aspects are described below. But it will of course be appreciated that in the development of any such actual embodiment, numerous implementation-specific decisions must be made to achieve the developers' specific goals, such as compliance with system-related and business-related constraints, which may vary from one implementation to another. Moreover, it will be appreciated that such development effort might be somewhat complex and time-consuming, but would nevertheless be a routine undertaking for those of ordinary skill in the art having the benefit of this disclosure.

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Where ever the phrases “for example,” “such as,” “including” and the like are used herein, the phrase “and without limitation” is understood to follow unless explicitly stated otherwise.

The terms “comprising” and “including” and “involving” (and similarly “comprises” and “includes” and “involves”) are used interchangeably and mean the same thing. Specifically, each of the terms is defined consistent with the common patent law definition of “comprising” and is therefore interpreted to be an open term meaning “at least the following” and is also interpreted not to exclude additional features, limitations, aspects, etc.

The term “about” is meant to account for variations due to experimental error. All measurements or numbers are implicitly understood to be modified by the word “about,” even if the measurement or number is not explicitly modified by the word “about.”

The term “substantially” (or alternatively “effectively”) is meant to permit deviations from the descriptive term that do not negatively impact the intended purpose. Descriptive terms are implicitly understood to be modified by the word “substantially,” even if the term is not explicitly modified by the word “substantially.”

As used herein, “low level light therapy” (“LLLT”) is the use of light comprising a plurality of application parameters, such as emitted wavelength(s), pulse frequencies, duty cycle/pulse width, intensity, individual treatment duration, total treatment duration, number of individual treatments during a total treatment regimen, time between individual treatments in a total treatment regimen, first LLLT administration time from an event (such as a surgery), time-course dosage, maintenance treatments, with the goal of optimal treatment, management, and/or cure of wounds or tissue degeneration at or near the genital area of a female patient (both cisgender and transgender who has undergone sex assignment) in need of such treatment, management, or cure.

“LLLT” as provided herein includes light generated by “low power” lasers (or “cold lasers”) and that generated by LED, or a combination of both. LED or laser light having therapeutic effects can be generated in a variety of wavelengths suitable for use in the inventions herein.

A “laser” is a device that emits light through a process of optical amplification based on the stimulated emission of photons. As would be recognized, the term “laser” originated as an acronym for light amplification by stimulated emission of radiation. The emitted laser light is notable for its high degree of spatial and temporal coherence. Lasers are classified as Class 1, Class 2, Class 3, and Class 4 by the American National Standards Institute. Such classifications are detailed in publication ANSI 7136.1-2000, the disclosure of which is incorporated herein in its entirety by this reference. The present invention utilizes Class 1, 2 and 3 lasers as light sources for LLLT.

A “light-emitting diode” (LED) comprises a two-lead semiconductor light source that is a p-n junction diode that emits light when activated. When a suitable voltage is applied to the leads, electrons are able to recombine with electron holes within the device, releasing energy in the form of photons. This effect is called “electroluminescence,” and the color of the light (corresponding to the energy of the photon) is determined by the energy band gap of the semiconductor. LEDs are typically small and integrated optical components may be used to shape the light emitting pattern. LEDs can be provided that emit various wavelengths and intensities of light, thus making them suitable for the inventive light therapies herein. More detail about wavelengths suitable for use in the present invention is disclosed hereinafter.

Light guides suitable for use herein can be fabricated from moldable optical silicone resins, such as those made by Krayden® (Dow Corning, Corning N.Y.). Suitable flexible light guides can also be fabricated from polyurethane, as set out in U.S. Pat. No. 7,433,565, the disclosure of which is incorporated herein in its entirety by this reference. Flexible light guides can also be made of clear TPE (thermoplastic elastomer). Materials suitable for use as light guides can be molded into lengths appropriate for various LLLT devices of the present invention using known methods. Yet further, light guides suitable for use herein can comprise fiber optics.

Various wound definitions can be used herein. As described in US Patent Application No. 20070239232, the disclosure of which is incorporated herein in its entirety, wounds can be characterized in various ways. For example, “acute wounds” are those that heal normally within a few weeks, while chronic wounds are those that linger for months or even years.

Wounds can also be characterized by the stage of healing and the functional parameters therewith. The line of closure fills with clotted blood (hemostasis), and, following the progression, in order, of inflammation, proliferation and maturation. For non-chronic wounds, the wound typically heals within a few weeks. Wounds that heal by secondary union (or secondary intention) typically involve large tissue defects, with more inflammation and granulation. Granulated tissue is needed to close the defect, and is gradually transformed into stable scar tissue. Such wounds are typically large open wounds as can occur from trauma, burns, and pressure ulcers. While surgical wounds are typically stitched, or stapled shut, which reduces the burden on the wound closing functionality, a subsequent infection or wound geometry can result in granulation occurring. While such a wound may require a prolonged healing time, but will not necessarily be chronic.

Mucosal healing, as in a vaginal cavity, the female genital area and areas proximal thereto are mechanistically similar to that for the skin. However, associated scarring and wound closure rates are different in mucosal healing, possibly due to differences in apoptosis. If granulated tissue is torn or otherwise damaged during the healing process, bleeding can result. This is a common occurrence with sex reassignment surgery, as noted, even though the wounds are surgically closed. Moreover, it is common for women to reopen episiotomy wounds by causing the sutures to break during a bowel movement or the like. Longer healing times will increase the risk that sutures will open, with subsequent healing likely leading to the generation of greater amounts of scar tissue than would have happened if the wound had not opened in the first place.

In a “chronic wound,” normal healing is not occurring, with progress stalled in one or more of the phases of healing. A variety of factors, including age, poor health and nutrition, diabetes, incontinence, immune deficiency problems, poor circulation, and infection can all cause a wound to become chronic. Typical chronic wounds include pressure ulcers, friction ulcers, and venous stasis ulcers. Stage 3 and Stage 4 pressure ulcers are open wounds that can occur whenever prolonged pressure is applied to skin and tissues covering bony outcrops of the body. Chronic wounds are also categorized, according to the National Pressure Ulcer Advisory Panel (NPUAP) relative to the extent of the damage.

In broad constructs, the present invention comprises devices and methods use light therapy applied in therapeutic amounts to treat, manage, and/or cure a female patient in need of such treatment. The indications for which the patient can be in need of treatment for can include one or more of open wounds, closed wounds, or chronic wounds proximal to the female genital region. Such wounds can be intentionally created (e.g., by way of a surgery etc.), accidentally caused, or chronic in nature.

The inventor herein has determined that application of light therapy, such as LLLT, can enhance healing of wounds in the vaginal cavity and vulvar regions, such as is relevant during male to female sex reassignment, while also being effective in reducing post-operative pain and inflammation. Yet further, the present invention can be utilized to enhance blood flow in female genital tissue, both within the vaginal cavity and in and around the vulvar region. As such, the inventor herein has further determined that LLLT can be effective in “vaginal rejuvenation,” as well as in addressing infections or post-birth injuries to the vulva or vaginal region that can occur, such as in some vaginal deliveries. Areas proximal to the female genitalia, such as the bladder and the anal area can also be therapeutically treated with light therapy according to the devices and methods herein.

In a significant aspect, the LLLT devices of the present invention are configured to be wearable by a patient within the vaginal cavity and/or in the vulvar region, as well as areas proximal thereto, in a patient in need of treatment, and the LLLT can be applied with such devices over an extended period, as directed by a medical provider. Such wearability has been found by the inventor herein to improve treatment compliance and enhance the efficacy of light therapy in a patient in need of treatment over prior LLLT delivery methods to the female genital region, when such LLLT is applied in accordance with in conjunction with one or more treatment parameters described herein.

Moreover, the inventions herein enable application of a set of defined LLLT dosages over an extended period as applied by at least one inventive wearable device, where the dosage is defined at least in relation to the medical condition/indication for which the patient is being treated. In some aspects, the inventions relate to selection of one or more patient medical indications in need of treatment. The inventive devices and methods can also incorporate patient specific information into the definition and delivery of dosage to the patient as prescribed by a medical provider. The inventions herein can therefore also relate to identification of one or more patient-specific parameters, whereby information associated with such patient-specific parameters is incorporated into a patient LLLT treatment regimen. Patient feedback and compliance information can be generated for incorporation into the treatment regimen. The devices and methods herein can also allow LLLT treatment to be easily and timely monitored by a healthcare provider and, if applicable, modified during a treatment regimen, thereby enhancing patient outcomes and ensuring safety of treatments.

The inventive LLLT devices comprise at least one low level light source in operational engagement with at least one light guide configured to deliver a therapeutic amount of LLLT substantially proximal to one or more locations at or near to the female genital area (whether naturally occurring female genitalia or reconstructed as in a male to female sex reassignment surgery) in a patient in need of treatment and, optionally, any areas associated with healing of the surgical wounds or in tissue regeneration.

A variety of light wavelengths can be appropriate for LLLT female genital area treatments herein. Generally, light in the ultraviolet, various visible ranges, and infrared light are each, individually, indicated for treatment of a wound, injury or medical indication at certain stages and associated healing areas. Accordingly, at least one aspect of the invention herein comprises selection of at least one light wavelength range appropriate for treating of a female genital area wound or female genital area condition in need of treatment.

Various effective wavelengths of light associated with wound healing have been reported at generally being effective between wavelengths of about 600 to about 900 nm (see e.g., FIG. 17 of U.S. Pat. No. 8,784,462, the disclosure of which is incorporated herewith in its entirety by this reference). The specific wavelengths identified as most effective for healing were reported to be 620, 680, 760, and 820 nm. Blue light in the spectrum range of from about 450 to about 495 nm is known to be effective for disinfection. Since wound disinfection is also relevant to wound healing, light in this nm range is also contemplated with the LLLT genital area devices and methods of the present invention. Wavelengths in the UV spectrum can also be effective in disinfecting a wound when signs of infection is detected or confirmed by the healthcare provider. The various wavelengths can be generated by a light source as would be known to one of ordinary skill in the art or can be determined through use of the inventive devices herein without undue experimentation by one of ordinary skill in the art.

In significant aspects, the inventive female genital area LLLT delivery device can comprise an insertable device configured to fit in the vaginal cavity where the device is substantially comprised of optically clear polymeric material, such as silicone, acrylic, or polycarbonate. Such medical grade materials are “hypoallergenic.” Optical clarity allows light of desired wavelength(s) and dosage(s) to travel from a light source in and through the device by way of one or more light guides for transmission to the vaginal cavity area for LLLT treatment thereof. “Optically clear” means that substantially all of the light emitted from the at least one light guide is then transmitted out of the surface of the therapeutic device. This means that there is substantially no loss or low loss of light as emitted from the at least one light guide into the interior of the vaginal insert so as to allow delivery of light having sufficient energy to provide a therapeutic effect to the patient's vaginal cavity and areas proximal thereto.

In some aspects of the invention, at least one light scattering layer can be created on the surface of the transparent core or an additional material can be embedded in the vaginal insert to enhance light diffusion. Such “discontinuities” are described in more detail hereinafter in relation to the Figures.

The size of the vaginal insert can be varied depending on the physical parameters of the patient being treated. The provider can measure, either subjectively or objectively, the approximate size of the patient's vaginal cavity. The provider will select the vaginal insert as sized for the patient being treated with LLLT.

As would be recognized, the outer surface of the vaginal cavity insert should be substantially approximate to the dimensions of the reconstructed vaginal cavity in a sex reassignment or the dimensions of the cisgender patient's vaginal cavity for use in a rejuvenation treatment or other suitable indications.

For a reconstructed vagina in a sex reassignment procedure, also called a “neovagina,” the overall length of the vaginal insert may have a smaller variation than that of a cisgender female because the vaginal cavity is being constructed from surgical norms. This is due to the limitations of the male body parts that are used to create the reconstructed vagina. In this regard, the depth of a typical neovagina created from male to female sex reassignment surgery is generally be between about 11 and 12 cm, as provided by the parameters of Dennovilliers' fascia, from which the neovagina is partially constructed. This is within the range of the natural female vagina.

For a cisgender female, the range of vaginal cavities may be greater. A 1996 study of the vaginas of 39 Caucasian women, found the following ranges of dimensions: lengths: 6.9 to 15 cm; widths: 4.8 to 6.3 cm; and introital diameters: 2.4 to 6.5 cm. A 2003 study, measured vaginal surface areas ranging from 66 to 107 cm² with a mean of 87 cm² and a standard deviation of 7.8 cm². Research published in 2006 gave the following mean dimensions, based on MRI scans of 28 women: mean length from cervix to introitus: 6.3 cm, and mean width: at the proximal vagina: 3.3 cm; at the pelvic diaphragm: 2.7 cm; and at the introitus: 2.6 cm. Thus, it can be observed that there is a potential for wide variation in the vaginal cavities of the cis gender females being treated.

Accordingly, the vaginal insert may be provided in a variety of lengths of from about 4 cm to about 15 cm and the diameter at the widest point can be from about 2 cm to about 10 cm. The shape of the vaginal insert can generally be cylindrical. For comfort, the interior end can be rounded. The insert can generally have a tapered configuration, as indicated by the general shape of a natural vaginal cavity, that is, wider at the anterior end (i.e., the part of the vaginal insert proximal to the vaginal opening), with a gradual reduction in the vaginal insert diameter moving toward the interior end (i.e., the part of the vaginal insert that is proximal to the internal end of the vagina, which would be the cervix in a cisgender woman, but would be absent in a male to female sex reassignment patient).

An advantage of the present invention is the wearability, comfort, safety and conformability to the treated vaginal cavity area. Moreover, treatment times, including dilation without applied light therapy, can be extended and/or made more convenient. The patient experiences enhanced wearability during treatment because the self-contained, that is, on board power, controller, light source(s), and, optionally, sensors allow concealment of the device during use and thus, enhanced privacy and comfort.

Application of LLLT to the vaginal cavity via an insertable device that is wearable by the patient for an extended period, can improve the healing process during the phases of wound healing. Treatment compliance can also be enhanced. To facilitate removal and insertion, the vaginal insert can incorporate a rim or tab integrally formed with the vaginal insert proximate to the anterior end thereof. The rim is sized and shaped to be comfortable for the patient, but to still enable the use thereof. To this end, the inventor herein has found that the rim or tab can be generally shaped as a teardrop, which has been found to comprise a balance between function and comfort for the patient. However, the size and shape of the rim and tab can be varied, or even not included on the vaginal insert, without departing from the scope and content of the invention. In some aspects, at least some of an anterior end of the vaginal insert can be situated outside the vaginal cavity. In some aspects, the controller and associated light guide(s) can be located within the structure of the vaginal insert, that is integrated within the vaginal insert itself, such as at or near the anterior end thereof. In other aspects, the controller and associated light guides can be present as a separate set of componentry for engagement with the vaginal insert.

In the case of rejuvenation or enhancing the internal structure of the vaginal cavity, the inventive LLLT devices and methods can enhance the effectiveness of such therapies by the applied LLLT stimulating the tissue, and thus growth of new, more elastic tissue, thereby tightening and enhancing feeling in the vaginal cavity area of a woman in need of treatment. Such treatment can also enhance the function of the female bladder by increasing muscle tone of areas proximal thereto.

Moreover, LLLT/dilation combination therapy used in conjunction with a light therapy delivery liner can substantially improve the long term outcomes of internal and external structures of a reconstructed external female genital areas (i.e., the vulvar area), by enhancing wound healing over prior art methodologies. Use of the light therapy delivery liner can improve wound healing of the vulvar region when used alone, such as would be indicated with an episiotomy, for example. Extended lengths for the light therapy delivery liner can allow treatment of hemorrhoids along with the vulvar areas, or hemorrhoids can be treated without attendant treatment of the vulvar areas. Moreover, LLLT light therapy applied both internally and externally to the vulvar regions can also be useful to treat vaginal fungal infections, and the methods and devices of the present invention also incorporate this treatment modality.

The light therapy delivery liner can be sized and shaped for the comfort of the patient. In this regard, the liner can generally be narrower at the front end (i.e., the area proximal to the clitoral area of the female genitalia) and wider at the back end. The corners are optimally rounded, again to enhance comfort. The shape can also approximate that of a panty liner or sanitary pad, as appropriate. To accommodate the various sizes of patients who will be treated with LLLT provided from the liner, the delivery liner can be provided in a variety of sizes. Generally, the length can be from about 20 to about 35 cm and the width can be from about 8 to about 16 cm at the widest end (i.e., in the area proximal to the rectum) and about 1 to about 10 cm at the narrowest part (i.e., in the area proximal to the clitoral area). If additional areas of the external female genitalia are being treated, the size and shape of the liner can be modified to enhance comfort. Soft and flexible materials should be used, such as silicone that has a Shore hardness of from about 0 to about 30, or from 15 to about 25.

The light therapy delivery liner can incorporate an indentation sized and shaped to allow the anterior end of the vaginal insert to engage therein. When a patient is wearing both the vaginal insert and the liner simultaneously, such engageable fit can improve comfort and wearability, especially when the liner is kept in place by well-fitting undergarments.

In addition to application of a therapeutic amounts of LLLT in an area substantially proximal to the internal and/or external female genital area wounds or conditions in need of treatment, in some aspects, the devices of the present invention further incorporate light treatment of patient areas that are in locations on the patient that are not the actual wound area. In this regard, the delivery element can further include application LLLT in “healing vital areas,” that is, areas associated with healing of the wound area, such as blood vessels, lymph nodes, nerves or acupuncture pressure points. Such areas can be those on the patient that are therapeutically associated with wound healing functions or tissue regeneration in the female genital regions.

Yet further, the LLLT delivery device can incorporate additional LLLT in a location away from the wound area where the additional LLLT is provided at an area proximal to those one or more areas of the patient that are associated with increasing blood flow to the area proximal to the wound or desired tissue regeneration. It is known that infrared and red light beams will penetrate to various depths, depending on wavelength (for example, the 830 nm wavelength will penetrate to a depth of nearly 5 cm). The laser beam at a low power (under 200 milliwatts [mW] or so) will not noticeably heat the soft tissue that it reaches, even with continuous use. When the beam hits the cells that line small arterioles, nitric oxide (a vasodilator) is released, thereby increasing local blood flow. Additionally, the laser will “desensitize” local nociceptors, thereby decreasing or even eliminating pain at the site. There is also evidence that light therapy can decrease inflammation, especially chronic inflammation. Similar results have been found with use of LEDs in various studies, as discussed previously.

The at least one (or one or more) light guides can be configured to enhance patient comfort, and thus wearability and patient compliance. In this regard, the at least one light guide is configured proximal to the anterior end of the vaginal insert, whereby the at least one light guide entry into the vaginal insert is at a position on the vaginal insert that is external to the vaginal cavity when the vaginal insert is in the patient's vaginal cavity. To further enhance patient comfort, the surface of the vaginal insert is substantially smooth.

One or more optical guides are utilized with the light therapy delivery liner. The optical guides are engaged with the liner to enhance wearability and patient comfort. In this regard, the optical guides can be engaged with the liner at a front or a back end.

It has been found by the inventor herein that LLLT effectiveness for both male to female sex reassignment, vaginal rejuvenation, as well as other therapeutic effects can be improved when the therapeutic light is emitted from the vaginal insert and/or as generated from a light source that can provide a known and/or regulated dosage, as opposed to being provided from one or more light sources that do not provide a known and/or regulated dosage of light. Therefore, the present invention comprises an insert and/or liner from which LLLT is provided in a known or regulated dosage amount, such as by a dosed light source.

The applied LLLT dosage can be as defined elsewhere herein. The LLLT dosage is defined by a medical provider for delivery to a patient in the amounts and periodicity so prescribed. The dosage is dispensed, and optionally can be monitored by, the controller that is in operational engagement with the light source, thereby making it a “dosed light source.” The controller is generally a microprocessor operated component, that can have firmware engaged therewith, or that can enable software instructions to be incorporated therewith, as are known. Dosage instructions, and optionally monitoring of the applied dosages, can be stored onboard the controller. The controller and/or any associated componentry can incorporate onboard communications capability, such as WiFi, Bluetooth®, RFID, etc.

A implementation of vaginal light therapy insert and light delivery liner of the present invention can comprise no exposed electronic circuitry, thereby allowing disinfecting liquid immersion for thorough sterilization to help prevent infection. Such contained electronic circuitry can also reduce the risk of electrical shock to the patient, thus further improving comfort and safety.

In this regard, the dosing light source, which can operate one or both of the vaginal insert and or liner, is configured to dispense a known or regulated dose of LLLT to a patient in need of treatment thereof. In some aspects, when both the liner and insert are being used, two controllers can be used. In other aspects, one controller can be used to operate both the liner and the vaginal insert LLLT dosage delivery.

It has been found by the inventor herein that LLLT effectiveness for both male to female sex reassignment and vaginal rejuvenation can be improved when the therapeutic light is emitted from the vaginal insert as generated from a light source that can provide a known and/or regulated dosage, as opposed to being provided from one or more light sources that do not provide a known and/or regulated dosage of light. Therefore, the present invention comprises an insert from which LLLT is provided in a known or regulated dosage amount, such as by a dosed light source.

The wavelength(s) of light applied to the female genital regions in need of treatment (that is vaginal cavity and/or vulvar areas and, optionally, areas proximal to these areas) can be the same or different from the wavelength(s) of light applied to areas proximal to areas that are associated with enhancement of wound healing of the genitalia. If the wavelengths applied to the different areas are the same, a single light guide can be configured to emit light proximal to the wound and the additional area(s). If the applied wavelengths are different, it can be beneficial to deliver the respective wavelengths with two different light guides. One or more light sources can be used to deliver the singular or multiple wavelengths through one or more light guides.

In further aspects, light reflected or emitted from vaginal cavity tissues while the vaginal insert device is inserted therein can be collected in one or more light guides operationally engaged with the vaginal insert for evaluation of such reflected or emitted light in a suitable sensor associated with the controller, such as photodetector with optical filters for desired wavelengths or the like. For example, it is known that during healing wounds may reflect or emit light in different wavelengths that can indicate the phases of healing. Further, wounds that are not healing correctly can reflect or emit light in wavelengths that can provide information about such inadequate healing progress or presence of infection. The inventions herein can be configured to allow such reflected or emitted light to be collected in one or more light guides in operational engagement with an evaluation device suitable for measuring the wavelength of light so reflected or emitted. Such detected light energy from the wound during healing can provide timely and accurate information and can be utilized to determine the healing progress of any wound healing, including in the female genital area undergoing treatment with the LLLT treatment devices herein. If the reflected or emitted light and associated evaluation thereof indicates that healing is occurring faster or slower than expected, the LLLT dosages can be timely modified, or the treatment stopped if appropriate. Such sensor data and measurement of healing progress can be desirable in near-real time monitoring the healing progress within a vaginal cavity because the patient may be highly sensitive during healing, and timely adjustment of treatment may reduce pain while the patient is being treated at home before a thorough in-office patient evaluation by a medical provider. Light reflected or emitted from the vulvar regions can also be evaluated, although it will be easier for a provider to visually evaluate the healing progress of such external genital areas.

The drawings presented below are intended to incorporate an expanded definition of female anatomy, that is, both that of cisgender females “naturally occurring” genitalia and transgender females who have undergone a male to female sex reassignment surgery. The described devices and methods can also be used for sex reassignment, vaginal rejuvenation and other indications related to the female genital areas where LLLT can be beneficial, sometimes with modification of the treatment regimens, as indicated.

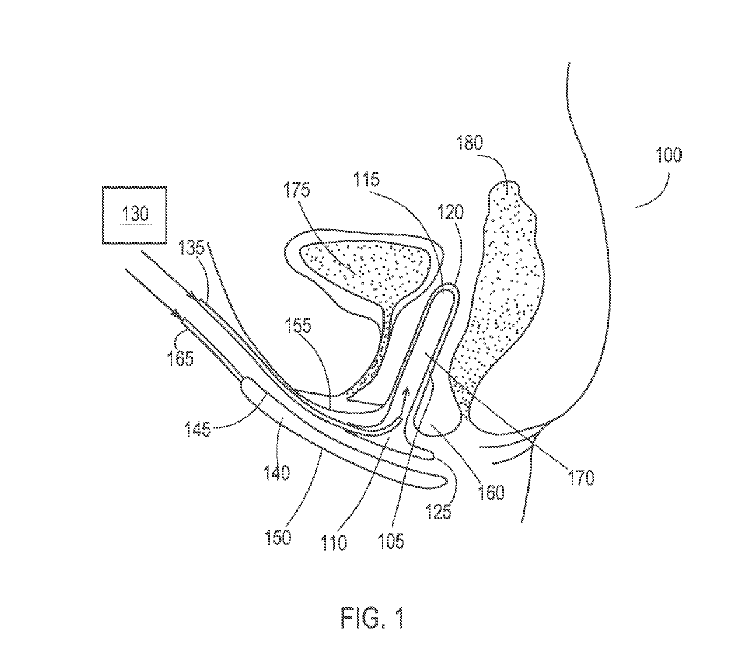

Referring to FIG. 1, female body 100, in the case of an illustrated male-to-female sex reassignment surgical patient, will normally require regular dilation to prevent collapse of the newly constructed vaginal cavity 105, among other medical requirements. In this regard, the inventive vaginal light therapy insert 110 can provide enhanced post-surgical healing. The present invention includes a vaginal light therapy insert 110, which can be fabricated of medical grade, optically transmitting clear material such as acrylic, polycarbonate, or silicone.

Vaginal light therapy insert 110 has interior end 115 that is configured to reach proximal to the interior end 120 of vaginal cavity 105. Vaginal light therapy insert 110 has anterior end 125 that is configured to facilitate removal of vaginal light therapy insert 110 from vaginal cavity 105, as necessary.

Vaginal light therapy insert 110 is operationally and optically engaged with dosing light source 130 via at least one light guide 135 for transferring light into vaginal light therapy insert 110 for subsequent transmission to vaginal cavity 105 etc. Dosed light source 130 can include a laser or LED light source [not shown] that emits light in the blue, green, red to near-infrared wavelengths for various stages and indications. Dosed light source 130 is operationally engaged with a controller [not shown] to regulate the power level, wavelength, duty cycle, pulsing frequencies to deliver desired dosages.

For example, light at about 650 nm to about 700 nm for red and about 830 nm to about 980 nm in the infrared range can be introduced to the vaginal cavity 105 at a dosage of from about 0.5 to about 1 Joule/cm²/day immediately post-surgery to promote healing during the early stages of recovery and to reduce pain and swelling in a male to female sex reassignment patient. The wavelengths associated with green, from about 510 nm to about 530 nm, can be administered at from about 0.5 to about 2.5 Joule/cm²/day, intermittently or simultaneously with other wavelengths, or can be used for treatment of vaginal or vulva rejuvenation, overactive bladder (OAB) syndrome, vaginal dilation, vaginismus or vulvodynia, conditions that can be associated with patients other than sex reassignment patients needing post-surgical healing assistance. The wavelengths associated with blue, from about 410 nm to about 495 nm, can be used at about 10 to about 90 mW/cm² to an actually or potentially infected area during wound healing.

Vaginal light therapy insert 110 can be incorporated in vaginal cavity 105 for extended periods to enhance healing thereof and to reduce the possibility that vaginal cavity 105 will collapse, such as is an adverse post-surgical complication in male to female sex reassignment, for example. Operationally, when vaginal light therapy insert 110 is inserted into vaginal cavity 105 during dilation—that is, post-operatively—the dimensions of vaginal light therapy insert 110 are configured to provide the effect of dilation. For vaginal rejuvenation, vaginal light therapy insert is also appropriately sized for therapeutic effectiveness and patient comfort.

Vaginal light therapy insert 110 can also be used in conjunction with vulvar area light therapy delivery liner 140 having first and second sides 145 and 150, respectively. First side 145 is configured to, for example, to deliver light to vulvar regions 155 and 160 of patient 100. Light therapy delivery liner 140 can be removed as needed for patient 100 to urinate etc.

Light therapy delivery liner 140 is operationally and optically engaged with dosing light source 130 via at least one light guide 165. The light source(s) [not shown] that are each, independently, engaged with vaginal light therapy insert 110 and light therapy delivery liner 140 from within dosing light source 130 can be the same or different. Light therapy delivery liner 140 can also be used alone as indicated by a medical provider in some cases, such as with perineum tears that can occur in vaginal births.

When dosing light source 130 is activated, LLLT is delivered through at least one light guide 135 to interior device portion 170 and/or through first side 145 so to allow light energy reach vaginal cavity 105 and vulvar regions 155 and 160.

Light therapy delivery liner 140 is optimally sized to maintain the comfort and privacy of patient 100 during healing. Wearability of light therapy delivery liner 140, as well as vaginal light therapy insert 110, can be enhanced by patient's 100 wearing of undergarments [not shown] that can keep light therapy delivery liner 140 in place which, in turn, can further secure light therapy delivery liner 140 in vaginal cavity 105.

LLLT dosages can be applied to either or both vaginal cavity 105 and vulvar regions 155 and 160 as prescribed by a medical provider. In this regard, and as non-limiting examples thereof, for sex reassignment surgery, the LLLT dose provided to a patient in need of treatment can have a duty cycle that is provided to from about 8 minutes on and about 30 minutes off for about three cycles per treatment on day 1, day 2 and day 3 postoperative, then drop to 2 cycles per treatment on about day 4 to about 8 post-op, thereafter one cycle per treatment until about day 24 for a sex-reassignment patient, for example. Moreover, the light source(s) [not shown] can provide light to either or both of vaginal cavity 105 or vulvar regions 155 and 160 at substantially the same times and wavelengths, or light LLLT treatment wavelengths or dosages can vary.

For vaginal rejuvenation, overactive bladder conditions, hemorrhoids or the like shorter periods of use, such as once or twice a day to achieve a dosage of from about 0.3 to about 3.5 Joule/cm²/day inside the vagina to provide therapeutic benefits to a bladder 175 or hemorrhoids proximal to the anal area 180 for a defined period, such as about 30 minutes, about 60 minutes or about 120 minutes per day may be indicated by the medical provider depending on the individual patient, however, variation of treatment times will not modify the inventive effects of the invention.

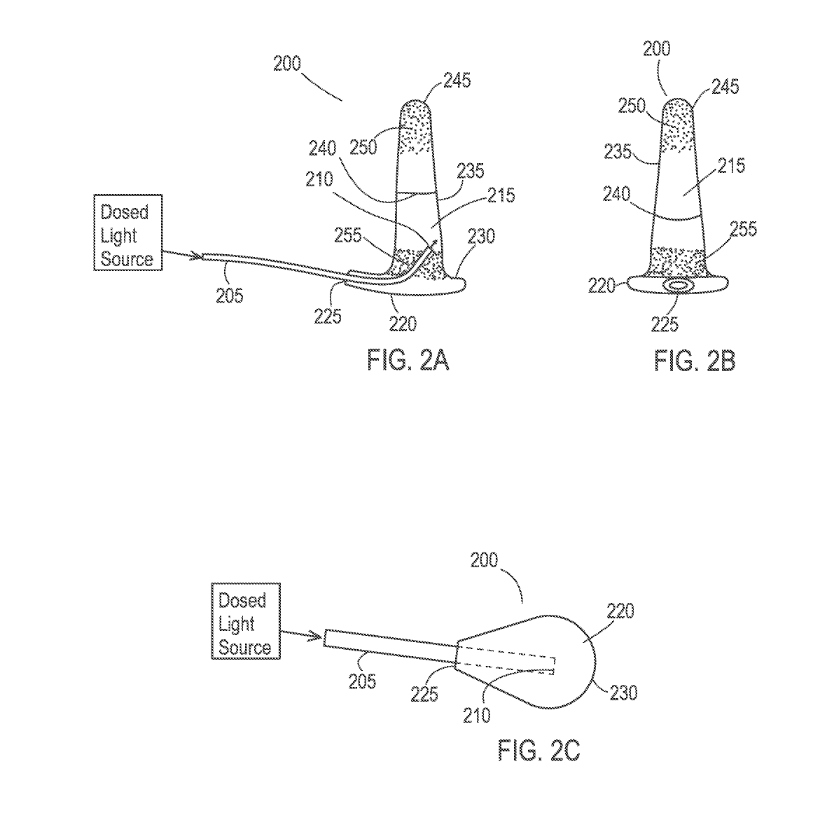

FIGS. 2a, 2b, 2c illustrate first, second, and third views of vaginal light therapy insert 200, wherein Vaginal light therapy insert 200 is rotated 90 degrees between 2a and 2b, and 2c is a bottom view thereof. Vaginal light therapy insert 200 is capable of both dilation and light energy delivery as shown. Light guide 205 is optically engaged with vaginal light therapy insert 200 by incorporation of light guide end 210 into interior 215 of vaginal light therapy insert 200 proximal to vaginal light therapy insert anterior end 220 at vaginal light therapy insert engagement port 225. FIG. 2b omits light guide 205 to better illustrate engagement port 225.

According to one aspect of the present invention, vaginal light therapy insert 200 can be configured having a rim 230, here shown, as one example, in the approximate shape of water drop, whereby rim 230 will substantially prevent the totality of vaginal light therapy insert 200 from wholly entering vaginal cavity 105 when inserted therein, while also making vaginal light therapy insert 200 easier to handle and extract. Vaginal light therapy insert 200 can be configured as an elongated portion 235 with outer surface 240 that together substantially conform to a diameter of a patient's vaginal cavity 105 with a small amount of taper from anterior end 220 to interior end 245. Various lengths and diameters of vaginal light therapy insert 200 can be configured as needed for each patient, as discussed in more detail herein.

In one aspect of the present invention, vaginal light therapy insert 200 can be fabricated from optically clear, medical grade polymers. To selectively allow a therapeutic dosage of LLLT to be emitted from vaginal light therapy insert 200, interior region 215 of vaginal light therapy insert 200 that is proximal to outer surface 240 (where the outer surface 240 is that aspect of vaginal light therapy insert 200 that comes into contact with the outer portion of patient vaginal cavity 105) can incorporate particles or added imperfections to generate discontinuity areas in one or more locations in vaginal light therapy insert 200. Such discontinuity areas, here shown as 250 and 255 have been found, in some aspects, to enhance light scattering light from interior 215 so as to improve the distribution of light to patient tissues [not shown] in use.

For example, surface discontinuity can be in the form of 255, which is configured to enhance light delivery at the entrance of vaginal opening area [not shown]. Clinically, with a sex reassignment surgery that incorporates a penile flap to provide a portion of a vulvar region in a patient, discontinuity area 255 has been found to facilitate the light concentrated radiation at the end of the vaginal opening area to reduce the occurrence of a post-surgical complication called “contracture.” Moreover, when a patient's colon flap is used in a male to female sex reassignment, discontinuity area 255 can also increase the light intensity at the area of opening of a patient's vagina, reducing the complication called “ring scar contraction” around the vaginal opening area. Incorporated air bubbles or particles inside vaginal light therapy insert 200 can also be used to create discontinuity area 250 while surface imperfections can be used to create discontinuity area 255, and vice versa.

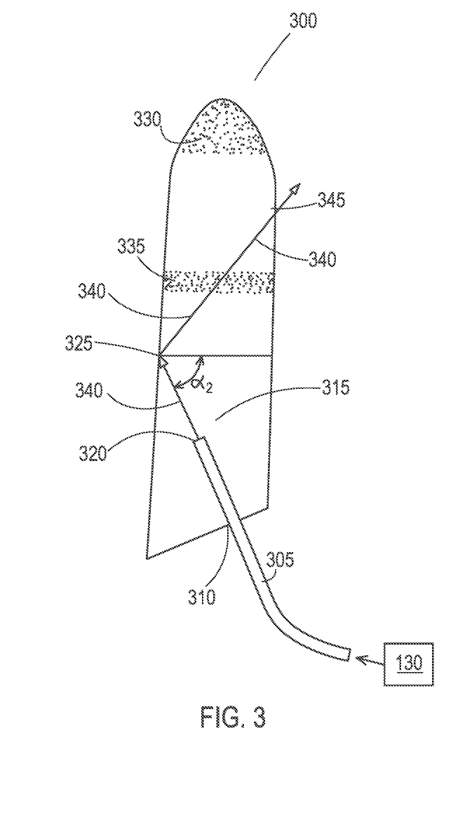

FIG. 3 illustrates vaginal light therapy insert 300 configured to minimize light loss so as to maximize light energy delivery to patient tissue needing treatment thereof when vaginal light therapy insert 300 is inserted in a vaginal cavity 105. Light guide 305 is incorporated within and is operationally and optically engaged with vaginal light therapy insert 300 through anterior end 310. Light guide 305 terminates at an interior device portion 315 of vaginal light therapy insert 300 such that light exiting light guide 305 at terminal end 320 enters interior device portion 315 to generate a light dominant-path, where more than 50% of light energy travels, at an optimum angle α2, which is configured to be greater than a critical angle for material from which vaginal light therapy insert 300 is constructed. Variations of light guide 305 placement and the resulting light transmission can be determined for a specific indication and/or patient need can be discerned by one of ordinary skill in the art without undue experimentation. As discussed previously, discontinuities can be incorporated into vaginal light therapy insert 300, shown here as 330 and 335.

If vaginal light therapy insert 300 is fabricated from a polycarbonate material with a refractive index of 1.6, for sections of vaginal light therapy insert 300 outside of vaginal cavity 105 when a majority of vaginal light therapy insert 300 is inserted in vaginal cavity 105, the refractive index of air, the critical angle of acrylic to air is about 38 degrees.

As would be understood by one of ordinary skill in the art, a critical angle is the angle of incidence above which total internal reflection occurs. By configuring light guide 305 to comprise a dominant light path angle α2 to be greater than about −38 degrees, the light entering interior device portion 315 will substantially be reflected at interior surface 325 so as to allow light beam 340 to travel within and through interior device portion 315 of vaginal light therapy insert 300 to exit vaginal light therapy insert 300 at 345, for example. Such an arrangement has been found by the inventor herein to substantially minimize light energy loss from the portion of vaginal light therapy insert 300 that is not located inside of the vaginal cavity 105 during use.

It should be noted that in this Figure, as in the other Figures accompanying this application, single light beams are shown for simplicity. However, the beams will be understood to comprise a range of angles associated with light emanating from the light guide and/or the tissues being treated with light therapy to provide therapeutic effects to a patient in need of treatment. Thus, light emanating from the light guides, traveling through the interior of the devices, exiting the devices for treatment of tissues, and emanating from tissues for capture by sensors should be understood to comprise diffuse light that will result in treatment of a range of tissue areas, as opposed being a single beam applied to a tissue area as might be inferred from the accompanying drawings.

Yet further, using the same example of vaginal light therapy insert device 300 being fabricated from acrylic, a dominant light path angle α2 can be provided to be less than the critical angle of acrylic to water. In some aspects, the placement of light guide 305 in interior device portion 315 can be arranged to provide a light path configuration having the following formula to enhance the therapeutic benefits of light energy.

α critical A<α2<α critical B Light dominant path formula:

Where α critical A is the critical angle the vaginal light therapy insert 300 material to air and α critical B is the critical angle the vaginal light therapy insert 300 material to vaginal mucosa.

Referring to FIG. 3, in a further aspect of the present invention with optical arrangement conforming to the above formula, light beam 340 can travel along a dominant path upon exiting light guide 305 at 320. When used inside a vagina mucosa with a refractive index of about 1.3 with more than about 15% less than the refractive index of vaginal light therapy insert 300, total internal reflection of light becomes possible with a critical angle of about 54 degrees. Upon light beam 340 reaching surface 325, light beam 340 will be reflected back into interior device portion 315. When light beam 340 reaches insert surface 345, a portion of light beam 340 can enter the vaginal cavity tissue [not shown] for therapeutic effect. Configuration of the light guide 305 and associated light beam 340, in conjunction with the LLLT dosage provided from dosing light source 130 can substantially reduce loss of light energy in light beam 340 when traveling through interior device portion 315 for subsequent delivery to a patient. Alternatively, a cladding/scattering layer [not shown] can be incorporated over the surface 345 to further distribute the light substantially evenly over the mucosa tissue.

Alternatively, when light scattering cluster particles or air bubbles are introduced to vaginal light therapy insert device 300, α2 can deviate from the light dominant path formula:

α critical A<α2<α critical B.

For example, light beam 325 can be directed at a cluster of light scattering particles to generate a more randomized beam pattern for light delivery to the mucosa. Further, when the surface of vaginal light therapy insert 300 is covered by a cladding material [not shown] with refractive index different than α critical A or α critical B, α2 can be optimized for delivery to desired target tissue areas.

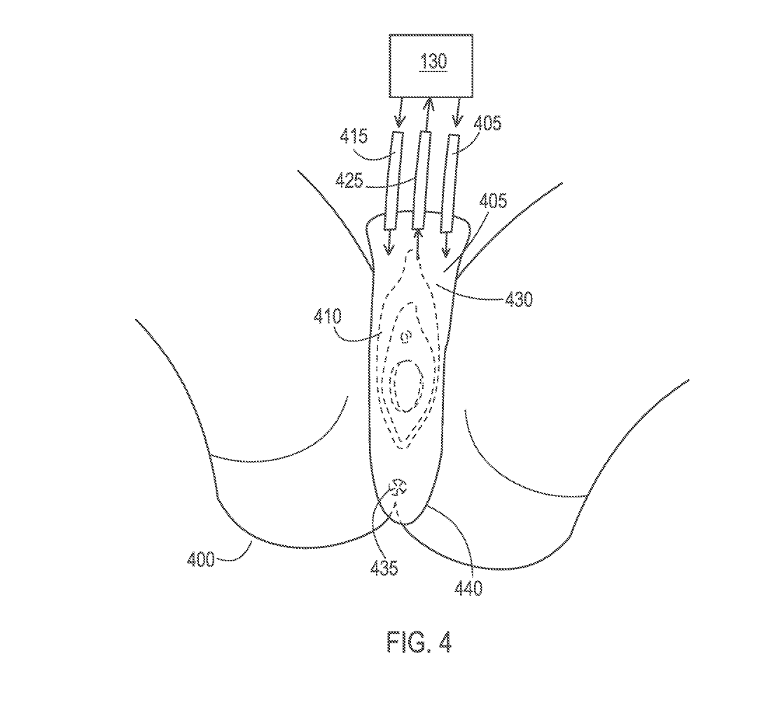

FIG. 4 illustrates the relative position of patient 400 to light therapy delivery liner 405 and the anatomic structure of at least vulvar region 410, shown here with dotted lines. Patient 400 is wearing light therapy delivery liner 405 that incorporates first light guide 415, optionally, second light guide 420 and third light guide 425, which can be used to deliver additional energy or light at a different wavelength than light delivered via 415, or to transmit light from the light therapy delivery liner 405 to associated sensors [not shown] for analysis. Light therapy delivery liner 405 has a first side 425 (not shown) proximal to vulvar region 410, from which light therapy is delivered thereto. First side 425 (not shown) can be in direct contact with vulvar region 410, or a bandage (not shown) or absorbent material (not shown) can be located between first side 425 (not shown) and vulvar region 410. As would be recognized, any such bandage or absorbent material should be light transmissive to allow light therapy to be suitably applied to vulvar region 410. Second side 430 will be proximal to the patient's undergarments etc. in use. Second side 430 is optimally configured to substantially prevent light from exiting therefrom, so as to minimize light loss. Accordingly, second side 430 can be coated with an optically opaque and reflective material. Still further, second side 430 can be coated or otherwise treated with a semi-reflective material.

Light guide 415 and (if present) light guides 420 and 425 are operationally and optically engaged with light therapy delivery liner 405 and dosing light source 130. When light therapy delivery liner 405 is placed proximal to vulvar and anal area, light energy can be delivered from dosing light source 130 to help accelerate wound healing resulting from sex reassignment surgery, laceration or episiotomy from baby delivery or other forms of injury, such as infection, which can occur in vulvar region 410, or hemorrhoids that can occur in anal region 435. Note that light therapy delivery liner 405 is shown with extended length to cover both vulvar region and anal region 435, however, if treatment is for the vulvar regions only, the length will be shorter. Shape and size can be selected to provide optimum fit for a patient, and the condition being treated.

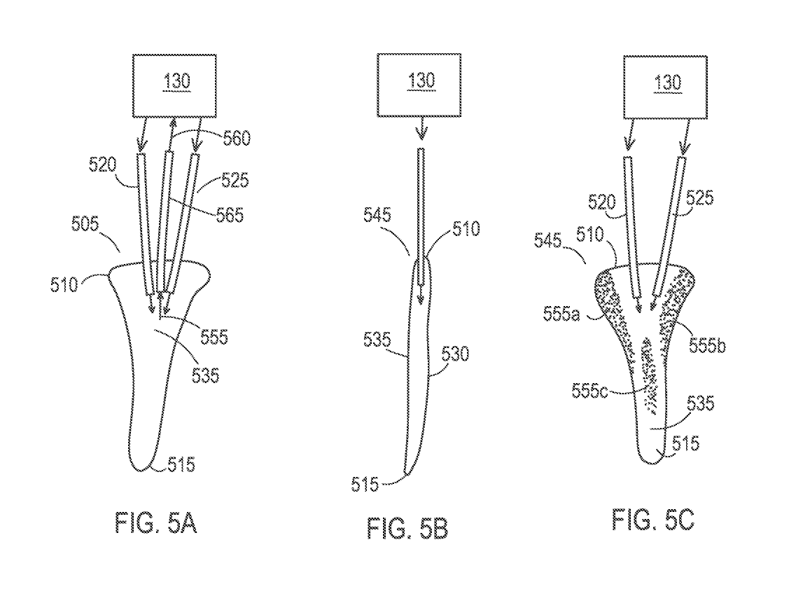

FIGS. 5a, 5b, and 5c illustrate light therapy delivery liner configurations that can be placed proximate to female vulva area to accelerate healing, reduce pain, and swelling. Referring to FIG. 5a, the size and shape of light therapy delivery liner 505 can be selected to conform to an identified patient vulvar region shape and size, such as having a wider rear portion 510 (which may be worn proximate to the patient's vulva region 410 or anal region 435 or shorter as needed for a specific therapeutic configuration) and a narrower front portion 515 (which may be worn proximate to the patient's clitoral region). Generally, light therapy delivery liner 505 can be configured to approximate an elongated triangle shape with a contoured edge so as to improve comfort and wearability. First side 530 (not shown), which is, in use, placed proximal to the patient's vulvar region 410 and, optionally, anal region 435, is configured to allow light to be delivered from dosing light source 130, where that light source is in operational and optical communication with light guide 520 and (optionally) light guides 525 and 565, which can be used to deliver additional energy or light at a different wavelength than light delivered by light guide 520, or to transmit light beam 555 from light therapy delivery liner 505 as beam 560 back to sensors [not shown] associated with dosing light source 130 for analysis to determine, for example, tissue condition, treatment progress, compliance and dose-response.

Wearabilty and patient comfort can be enhanced by using a softer (with Shore A Hardness of about 30 or less) material to fabricate light therapy delivery liner 505 for example. In one aspect of the present invention, Light therapy delivery liner 505 can be configured from optically clear silicone rubber with a Shore A hardness of greater than out 0 to about 30.

Referring to FIG. 5b, 545 is a side view of light therapy delivery liner 505, where first side 530 is shown as contoured along the length thereof, and first end 515 is shown terminating in a somewhat curved configuration so as to enhance comfort in wearability.

Referring now to FIG. 5C, light therapy delivery liner 550 incorporates surface or imbedded discontinuities 555a, 555b and 555c to enhance light scattering, which can be beneficial in some implementations. In one aspect of the invention, discontinuities can be provided by impregnating particles in the surface of light therapy delivery liner 550. In another aspect, discontinuities 555a, 555b and 555c can be generated by etching the mold with sand or other particle-type etching to achieve discontinuity configurations desirable for light scattering. Discontinuity areas 555a, 555b and 555c, as well as any other discontinuity configurations that might be appropriate for a patient treatment or device functionality, can be configured to increase or enhance the light exiting light therapy delivery liner 545 for treatment of vulvar tissues and, optionally, anal tissues. As an example of the selective scattering applications, discontinuity areas 555a and 555b are configured to provide light energy to the groin regions of the patient in use, especially to the area with lymph nodes and blood vessels near the pelvic area, which can function as “healing vital areas,” as discussed previously. Discontinuity area 555c can enhance healing of the area proximal to the vaginal opening.

As examples for treatment method to the vulva area with light therapy delivery liner 550, light can be introduced to the vulvar region 410 at a dosage of from about 0.3 to about 5 Joule/cm²/day with red wavelengths of from about 650 nm to about 700 nm and about 830 nm to about 980 nm in the infrared range immediately post-surgery or shortly after childbirth to promote healing during the early stages of recovery and to reduce pain and swelling. The wavelengths associated with green, from about 510 nm to about 530 nm, can be administered at about 0.3 to about 3 Joule/cm²/day, intermittently or simultaneously with other wavelengths, or can be used for treatment of vaginal or vulva area rejuvenation, overactive bladder (OAB) syndrome, vaginismus or vulvodynia, conditions that can be associated with patients needing therapeutic assistance. For an actually or potentially infected area during wound healing, the wavelengths associated with blue, from about 410 nm to about 495 nm, can be used with at about 10 to about 70 mW/cm².

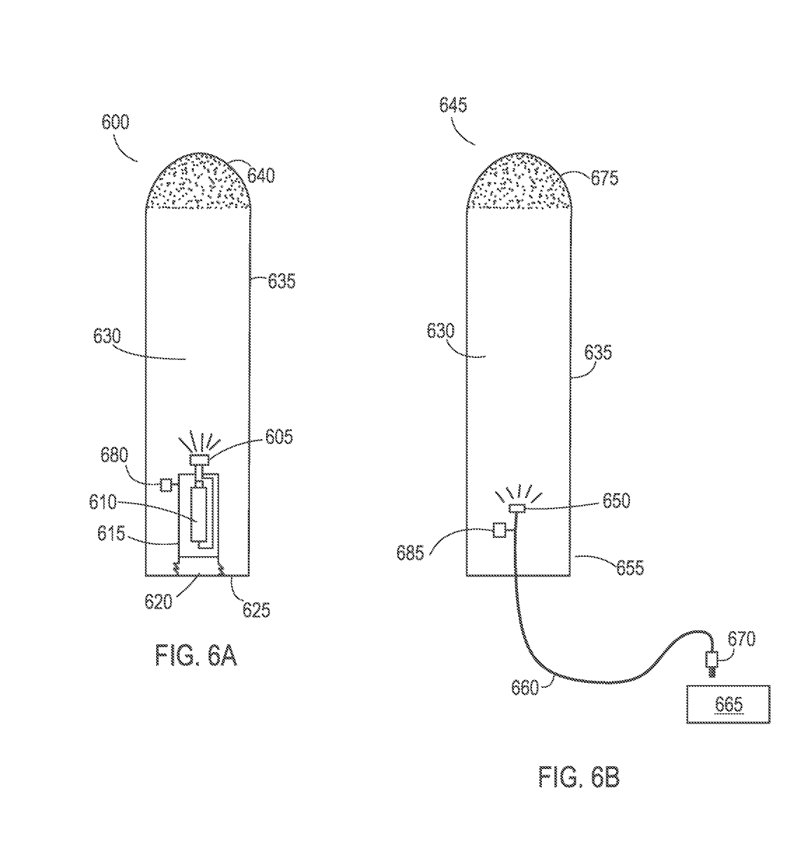

In another aspect of the present invention shown in FIG. 6a, vaginal light therapy insert 600 can be operationally configured with a light source 605, controller 610, associated housing 615 and removable battery 620 proximal to an anterior end 625 of vaginal light therapy insert 600. Vaginal light therapy insert 600 is configured from optically clear material. Light source 605 can be a LED or other suitable light source that is configurable emit a therapeutic wavelength of light. In use, light source 605 emits light with desired wavelength into and through the optically clear interior potion 630 to be emitted from outer surface 635. Discontinuities 640 can optionally be incorporated in one or more locations on vaginal light therapy insert 600 to enhance light scattering.

In FIG. 6b, Vaginal light therapy insert 645 incorporates a LED 605 or other suitable light source, LED 650 is operationally connected via wire 660 to controller 665, which is powered by or connected to battery power [not shown] and can have wireless communication capabilities [not shown] such as Bluetooth or WiFi. As illustrated, connection 670 is removably engagable with controller 665, but such connection can be permanent, as appropriate. Discontinuities 640 and 675 can be incorporated as discussed herein in any portion of the devices illustrated in FIGS. 6a and 6b. Vaginal light therapy inserts 600 and 645 each, independently, can further be configured with sensors 680 and 685 that are in operational engagement with the respective controllers 610 and 665.

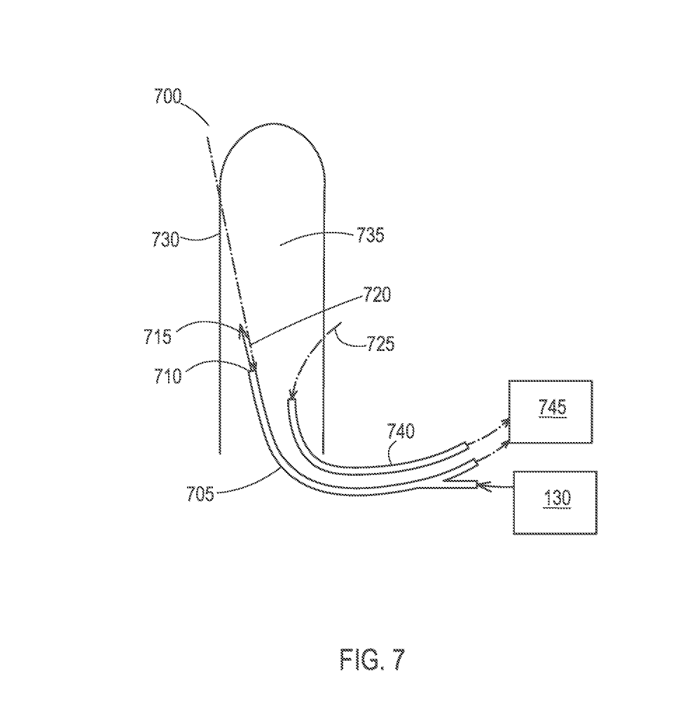

Referring to FIG. 7, Vaginal light therapy insert 700 is configured with light guide 705 from which therapeutic light is emitted at 710 as light beam 715, which provides therapeutic light when it reaches the patient's vaginal tissue [not shown]. Light guide 705 is in operational and optical communication with dosing light source 130. When inserted in a vaginal cavity [not shown] and the treatment process is underway, the patient's vaginal tissue [not shown] condition, such as temperature, infection, oxygen level, dryness, wetness, coloration, smoothness, and others can be analyzed in various methods, such as with reflected light, or emitted light of one or more wavelengths from bioluminescence or fluorescence associated with the healing process. Light detection from vaginal cavity 105 during healing, here shown for simplicity as tissue reflected or emitted beam 720 and tissue reflected or emitted beam 725 can travel through outer device surface 730 into interior 735 for collection by light guides 710 and 740. Such collected light beams 720 and/or 725 are transmitted to analysis device 745, which can be a photodetector or other suitable sensor-containing equipment, to evaluate the healing level and effectiveness of the relevant area. FIG. 7 is shown with two light guides, 705 and 740, where 705 can be used to both emit LLLT and to collect light from the vaginal cavity tissue, however, other configurations are contemplated.

Variations in the devices and methods herein can be provided without detracting from the scope of the present invention.

What is claimed is:

1. A therapeutic device comprising:
a. a structure having an interior end and an anterior end, wherein the structure is:
i. fabricated from an optically clear material;
ii. wider at the anterior end than at the interior end, thereby providing a tapered orientation for the structure; and
iii. engagable with at least one light guide, wherein the at least one light guide is operationally engaged with a light source configured to dispense one or more doses of low level light therapy (LLLT) to a vaginal cavity of a patient when a substantial portion of the structure is inserted in at least part of the vaginal cavity of the patient.

2. The therapeutic device of claim 1, wherein the patient is a male to female sex reassignment patient.

3. The therapeutic device of claim 1, wherein the patient is a cisgender female.

4. The therapeutic device of claim 1, wherein the interior end is rounded.

5. The therapeutic device of claim 1, wherein the at least one light guide is engaged with the structure at a location proximal to the anterior end of the structure, and the light guide is substantially exterior to the patient's vaginal opening when the structure is present in the vaginal cavity.

6. The therapeutic device of claim 1, wherein the light source comprises one or more lasers.

7. The therapeutic device of claim 1, wherein the light source comprises one or more LEDs.

8. The therapeutic device of claim 1, wherein a rim is located proximal to the anterior end, thereby facilitating removal of the structure from the vaginal cavity.

9. The therapeutic device of claim 1, wherein the LLLT dosage applied to the patient is generated by a controller in operational engagement with the light source and wherein the controller is configured to dispense LLLT according to dosage instructions generated by a medical provider.

10. The therapeutic device of claim 1, wherein the structure is fabricated from one or more of acrylic polymer, polycarbonate, or silicone rubber.

11. A therapeutic device comprising:
a. a flexible structure for use proximal to the genitalia of a female patient in need of low level light therapy (“LLLT”), wherein the structure has a first end and a second end, and a first side and a second side, wherein:
i. the first end is configured to fit proximal to a front genital area of the patient;
ii. the second end is configured to fit proximal to a rear genital area of the patient;
iii. the structure is engageable with at least one light guide, wherein the at least one light guide is operationally engaged with a light source configured to dispense one or more doses of LLLT to the patient; and
iv. the first side is optically clear,
thereby allowing LLLT emitted from an interior thereof to be dispensed to at least some of the genital area of the patient when the first side is worn by the patient with the first side facing the genital area of the patient.

12. The therapeutic device of claim 11, wherein the patient is a male to female sex reassignment patient.

13. The therapeutic device of claim 11, wherein the patient is a cisgender female.

14. The therapeutic device of claim 11, wherein the light source comprises one or more lasers.

15. The therapeutic device of claim 11, wherein the light source comprises one or more LEDs.

16. The therapeutic device of claim 11, wherein the second side of the structure comprises an optically reflective coating, and wherein the reflective coating is applied so that light contacting the coating will reflect light emitted from the at least one light guide in the direction of the first side.

17. The therapeutic device of claim 11, wherein the structure is fabricated from silicone that has a Shore hardness of greater than about 0 to about 30.

18. The therapeutic device of claim 11, wherein the at least one light guide is engaged at the second end.

19. The therapeutic device of claim 11, wherein the LLLT dosage applied to the patient is generated by a controller in operational engagement with the light source, and wherein the controller is configured to dispense LLLT according to dosage instructions generated by a medical provider.
